(19) United States
(12) Patent Application Publication
(10) Pub. No.: US 2026/0137362 A1
(43) Pub. Date: May 21, 2026

(54) Image Enhancement and Embedded Advanced Processing in Medical Imaging

(71) Applicant: MedCognetics, Inc., Plano, TX (US)

(72) Inventors: Scott Chase Waggener, Rockport, TX (US); Timothy Cogan, Frisco, TX (US); Debasish Nag, Richardson, TX (US); Lakshman Tamil, Plano, TX (US)

(21) Appl. No.: 19/396,187

(22) Filed: Nov. 20, 2025

Related U.S. Application Data
(60) Provisional application No. 63/722,777, filed on Nov. 20, 2024.

(51) Int. Cl. A61B 6/50; A61B 6/00; A61B 6/12; A61B 6/42; A61B 6/46

(52) U.S. Cl. CPC A61B 6/502; A61B 6/12; A61B 6/4258; A61B 6/461; A61B 6/5217; A61B 6/5264

(57) ABSTRACT

System and method enhances the quality of the raw image capture and applies artificial intelligence analysis on each step of the image enhancement process. An embedded processor includes or executes deep learning algorithms capable of delivering one or more image features by leveraging intermediate representations at any stage of the digital processing pipeline inside the imaging machine. The deep learning algorithm is composed of fully connected, attention, convolutional, or any layers performing linear and non-linear operations for feature extraction and signal processing, or a combination.

### CROSS-REFERENCE TO RELATED APPLICATIONS

This claims the benefit of U.S. patent application 63/722,777, filed Nov. 20, 2024, which is incorporated by reference along with all other references cited in this application.

### BACKGROUND OF THE INVENTION

This invention relates to automated mammogram classification and more particularly, to systems and methods for deep learning-based mammogram classification that are racially unbiased. In particular, this invention relates to embedded systems, and, more particularly, to systems and methods for image enhancement and deep learning-based algorithms which physically reside within medical imaging hardware.

Worldwide, medical imaging equipment renders radiographic/magnetic/sonic based images of patient organs, based premise of emission of a radiographic/magnetic/sonic signal that passes through a portion or the entire body of the patient, and the pass-through signal is detected, from which an image is formed. By the time the medical image is formed artifacts, distortions, signal scattering, and patient movement are also captured and are part of the medical image. Such a medical image, with said artifacts, makes it difficult for the radiologist or technicians to clearly see the portion of the patient's body that was exposed to the medical imaging equipment. Medical imaging equipment today are not tuned using AI and the image processing systems are not optimized using AI; they are not controlled by a dynamic, real-time AI embedded software system that can be upgraded.

For example, breast cancer is the most common cancer for women worldwide and the second most common cancer when considering both men and women. In addition, breast cancer is the leading cause of cancer death in women across the globe. For women as young as 40, mammograms, x-ray images of breast tissue, can provide a cost-effective means for breast cancer screening. However, consistent image quality varies from mammogram machine type and manufacturer which makes it difficult for radiologists to screen effectively and in a nominal manner. A software-defined image processing system can improve the raw image capture mammography rendition in various stages of an image development path or commonly known as an Image Pipe. Such methodologies have been used in camera technology in automotive safety systems, robotic machine vision for industrial applications, and in camera phone features in mobile devices.

Typically, mammograms are screened by radiologists who determine if a biopsy is needed to classify a tissue abnormality as malignant or benign. Image Processing and Embedded Machine learning algorithms capable of performing at or above the level of a radiologist could potentially replace or assist radiologists and thereby reduce the cost of mammograms as well as lead to earlier and more reliable detection of breast cancer.

All the deep learning networks developed so far in general operate external to the medical imaging device, as in this case, the mammogram imaging gantry. The image processing system and machine learning algorithms described here will now be able to operate within the medical imaging equipment and render enhanced image quality and deep-learning findings.

Therefore, there is a need for an improved system and method for image enhancement and embedding deep learning-based algorithms in medical imaging equipment.

### BRIEF SUMMARY OF THE INVENTION

A system and method enhance the quality of the raw image capture and applies artificial intelligence analysis on each step of the image enhancement process. An embedded processor includes or executes deep learning algorithms capable of delivering one or more image features by leveraging intermediate representations at any stage of the digital processing pipeline inside the imaging machine. The deep learning algorithm is composed of fully connected, attention, convolutional, or any layers performing linear and non-linear operations for feature extraction and signal processing, or a combination.

Further, the deep learning algorithm can denoise the image by removing optical artifacts, scatter noise, and any other unwanted signal. The deep learning algorithm can filter motion control to remove image blurring due to patient or body movement at the instance of image capture. Layers of a deep learning network extract features for the context of optimizing or correcting the position of the patient who is being imaged, or a combination.

The algorithm can predict the breast density from the image. Layers of a deep learning network extract image features for the context of a specific cancer or benign outcome for a triage case list for presentation to the radiologist. The algorithm can denote the region of interest (RoI) of cancer (e.g., tissue of interest) in the mammogram image. The algorithm can assess the risk of the patient getting breast cancer from the breast image and provide a risk score. The algorithm is capable of increasing image clarity and resolution without the physical increase of the projections or voxels in image capture. The algorithm can be based on projection synthesis using generative AI. The algorithm can also be based on voxel synthesis using generative AI.

In an implementation, a system includes: a medical imaging emitter device; a medical imaging detector device, where a tissue to be examined is to be placed between the emitter and detector devices or the tissue to be examined will be placed in between a signal path from the emitter device to the detector device; an analog front end circuit, connected to the detector circuit; an analog-to-digital converter (ADC) circuit, coupled to the detector circuit; a digital signal processing (DSP) circuit, connected to the analog-to-digital converter circuit; a system bus, connected to digital signal processing circuit; a processor, coupled to system bus; and an image processing circuit, connected to the system bus, where the image processing circuit includes: tensor cores or matrix-multiply-accumulate units, or both; a random access memory, connected to the tensor cores or matrix-multiply-accumulate units; a nonvolatile memory, connected to the tensor cores or matrix-multiply-accumulate units; and a network interface, connected to the tensor cores or matrix-multiply-accumulate units.

The digital signal processing (DSP) circuit can include or be implemented in a field programmable gate array. The image processing circuit can be configured to execute (i) image preprocessing, (ii) image enhancement, (iii) image analysis, (iv) analysis visualization, and (v) automated report generation. The image processing circuit can be configured to execute a fully connected, attention, convolutional, or any layers performing linear and non-linear operations for feature extraction and signal processing, or any combination. The medical imaging emitter device can be at least one of an x-ray emitter (as part of x-ray machine or CT scan machine), magnetic field emitter (as part of MRI machine), or positron emitter (as part of a PET scan machine).

Further, the image processing circuit can be configured to execute one or more deep learning algorithms capable of delivering one or more image features by leveraging intermediate representations at any stage of the digital processing pipeline inside the imaging machine. The image processing circuit can execute a technique including of fully connected, attention, convolutional, or any layers performing linear and non-linear operations for feature extraction and signal processing, or any combination. The image processing circuit can denoise the image by removing optical artifacts, scatter noise, and any other unwanted signal. The image processing circuit can filter motion control to remove image blurring due to patient or body movement at the instance of image capture.

Still further, the image of the tissue can be a mammogram, and layers of a deep learning network extract features for the context of an optimizing or correcting the position of the patient who is being imaged, or a combination. The image processing circuit can predict a breast density from the image. The image of the tissue can be a mammogram, and layers of a deep learning network extract image features for the context of a specific cancer or benign outcome for a triage case list for presentation to the radiologist. The image processing circuit can denote a region of interest (RoI) of cancer in a mammogram image. The image processing circuit can assess the risk of the patient getting breast cancer from the breast image and provide a risk score. The image processing circuit can increase an image clarity and resolution without the physical increase of the projections or voxels in image capture. The image processing circuit can increase an image clarity and resolution based on projection synthesis using a generative intelligence technique, without the physical increase of the projections or voxels in image capture. The image processing circuit can increase an image clarity and resolution based on voxel synthesis using a generative intelligence technique.

Other objects, features, and advantages of the present invention will become apparent upon consideration of the following detailed description and the accompanying drawings, in which like reference designations represent like features throughout the figures.

### BRIEF DESCRIPTION OF THE DRAWINGS

FIG. 1 shows a simplified block diagram of a client-server system implemented in a distributed computing network connecting a server and clients.

FIG. 2 shows a more detailed diagram of an exemplary client or computer which may be used in an implementation of the invention.

FIG. 3 shows a system block diagram of a client computer system used to execute application programs such as a web browser or tools.

FIG. 4a shows a system diagram describing the embedded image processing system in an imaging machine.

FIG. 4b shows a deep learning network embedded in a medical imaging device, in this example, a mammogram gantry system.

FIG. 5 shows component and block level view of an artificial intelligence embedded image pipe that resides inside medical imaging equipment, like a mammogram gantry system.

FIG. 6 shows artificial intelligence enhancements and methods applied to improve image quality of a medical image.

FIG. 7 shows artificial intelligence guided lesion-informed projection for image reconstruction from a raw image source in a medical device.

FIG. 8 shows artificial intelligence digital image processing for denoising, increasing resolution, and emphasizing lesion areas from a reconstructed image that is sourced from raw pixel data from a medical device.

FIG. 9 shows artificial intelligence driven presentation enhancement by using contrast enhancement and windowing methods on a presentation image within a medical device.

FIG. 10 shows artificial intelligence embedded features that can be applied along the image pipe.

FIG. 11 shows a complete embedded artificial intelligence system with enhanced image processing from raw image capture from the medical imaging device and artificial intelligence analysis of said processed medical images.

FIG. 12 shows a picture depicting the synthesis of projections using artificial intelligence.

FIG. 13 shows a picture depicting the synthesis of voxel projections using artificial intelligence.

FIG. 14 shows a system diagram of an imaging machine with embedded image processing system.

### DETAILED DESCRIPTION OF THE INVENTION

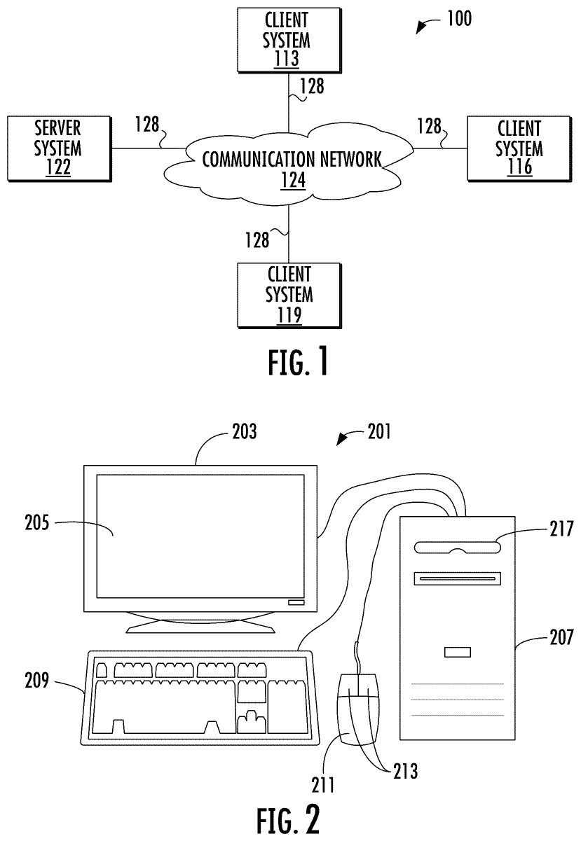

FIG. 1 is a simplified block diagram of a distributed computer network 100 incorporating an embodiment of the present invention. Computer network 100 includes a number of client systems 113, 116, and 119, and a server system 122 coupled to a communication network 124 via a plurality of communication links 128. Communication network 124 provides a mechanism for allowing the various components of distributed network 100 to communicate and exchange information with each other.

Communication network 124 may itself be comprised of many interconnected computer systems and communication links. Communication links 128 may be hardwire links, optical links, satellite or other wireless communications links, wave propagation links, or any other mechanisms for communication of information. Communication links 128 may be DSL, Cable, Ethernet or other hardwire links, passive or active optical links, 3G, 3.5G, 4G and other mobility, satellite or other wireless communications links, wave propagation links, or any other mechanisms for communication of information.

Various communication protocols may be used to facilitate communication between the various systems shown in FIG. 1. These communication protocols may include VLAN, MPLS, TCP/IP, Tunneling, HTTP protocols, wireless application protocol (WAP), vendor-specific protocols, customized protocols, and others. While in one embodiment, communication network 124 is the Internet, in other embodiments, communication network 124 may be any suitable communication network including a local area network (LAN), a wide area network (WAN), a wireless network, an intranet, a private network, a public network, a switched network, and combinations of these, and the like.

Distributed computer network 100 in FIG. 1 is merely illustrative of an embodiment incorporating the present invention and does not limit the scope of the invention as recited in the claims. One of ordinary skill in the art would recognize other variations, modifications, and alternatives. For example, more than one server system 122 may be connected to communication network 124. As another example, a number of client systems 113, 116, and 119 may be coupled to communication network 124 via an access provider (not shown) or via some other server system.

Client systems 113, 116, and 119 typically request information from a server system which provides the information. For this reason, server systems typically have more computing and storage capacity than client systems. However, a particular computer system may act as both as a client or a server depending on whether the computer system is requesting or providing information. Additionally, although aspects of the invention have been described using a client-server environment, it should be apparent that the invention may also be embodied in a stand-alone computer system.

Server 122 is responsible for receiving information requests from client systems 113, 116, and 119, performing processing required to satisfy the requests, and for forwarding the results corresponding to the requests back to the requesting client system. The processing required to satisfy the request may be performed by server system 122 or may alternatively be delegated to other servers connected to communication network 124.

Client systems 113, 116, and 119 enable users to access and query information stored by server system 122. In a specific embodiment, the client systems can run as a standalone application such as a desktop application or mobile smartphone or tablet application. In another embodiment, a “web browser” application executing on a client system enables users to select, access, retrieve, or query information stored by server system 122. Examples of web browsers include the Edge browser program provided by Microsoft Corporation, Firefox browser provided by Mozilla, Chrome browser provided by Google, Safari browser provided by Apple, and others.

In a client-server environment, some resources (e.g., files, music, video, or data) are stored at the client while others are stored or delivered from elsewhere in the network, such as a server, and accessible via the network (e.g., the Internet). Therefore, the user's data can be stored in the network or “cloud.” For example, the user can work on documents on a client device that are stored remotely on the cloud (e.g., server). Data on the client device can be synchronized with the cloud.

FIG. 2 shows an exemplary client or server system of the present invention. In an embodiment, a user interfaces with the system through a computer workstation system, such as shown in FIG. 2. FIG. 2 shows a computer system 201 that includes a monitor 203, screen 205, enclosure 207 (may also be referred to as a system unit, cabinet, or case), keyboard or other human input device 209, and mouse or other pointing device 211. Mouse 211 may have one or more buttons such as mouse buttons 213.

It should be understood that the present invention is not limited any computing device in a specific form factor (e.g., desktop computer form factor), but can include all types of computing devices in various form factors. A user can interface with any computing device, including smartphones, personal computers, laptops, electronic tablet devices, global positioning system (GPS) receivers, portable media players, personal digital assistants (PDAs), other network access devices, and other processing devices capable of receiving or transmitting data.

For example, in a specific implementation, the client device can be a smartphone or tablet device, such as the Apple iPhone, Apple iPad, Apple iPod, Samsung Galaxy product (e.g., Galaxy S series product or Galaxy Note series product), Google Nexus devices, and Microsoft devices (e.g., Microsoft Surface tablet). Typically, a smartphone includes a telephony portion (and associated radios) and a computer portion, which are accessible via a touch screen display.

There is nonvolatile memory to store data of the telephone portion (e.g., contacts and phone numbers) and the computer portion (e.g., application programs including a browser, pictures, games, videos, and music). The smartphone typically includes a camera (e.g., front facing camera or rear camera, or both) for taking pictures and video. For example, a smartphone or tablet can be used to take live video that can be streamed to one or more other devices.

Enclosure 207 houses familiar computer components, some of which are not shown, such as a processor, memory, mass storage devices 217, and the like. Mass storage devices 217 may include mass disk drives, floppy disks, magnetic disks, optical disks, magneto-optical disks, fixed disks, hard disks, CD-ROMs, recordable CDs, DVDs, recordable DVDs (e.g., DVD-R, DVD+R, DVD-RW, DVD+RW, HD-DVD, or Blu-ray Disc), flash and other nonvolatile solid-state storage (e.g., USB flash drive or solid state drive (SSD)), battery-backed-up volatile memory, tape storage, reader, and other similar media, and combinations of these.

A computer-implemented or computer-executable version or computer program product of the invention may be embodied using, stored on, or associated with computer-readable medium. A computer-readable medium may include any medium that participates in providing instructions to one or more processors for execution. Such a medium may take many forms including, but not limited to, nonvolatile, volatile, and transmission media. Nonvolatile media includes, for example, flash memory, or optical or magnetic disks. Volatile media includes static or dynamic memory, such as cache memory or RAM. Transmission media includes coaxial cables, copper wire, fiber optic lines, and wires arranged in a bus. Transmission media can also take the form of electromagnetic, radio frequency, acoustic, or light waves, such as those generated during radio wave and infrared data communications.

For example, a binary, machine-executable version, of the software of the present invention may be stored or reside in RAM or cache memory, or on mass storage device 217. The source code of the software of the present invention may also be stored or reside on mass storage device 217 (e.g., hard disk, magnetic disk, tape, or CD-ROM). As a further example, code of the invention may be transmitted via wires, radio waves, or through a network such as the Internet.

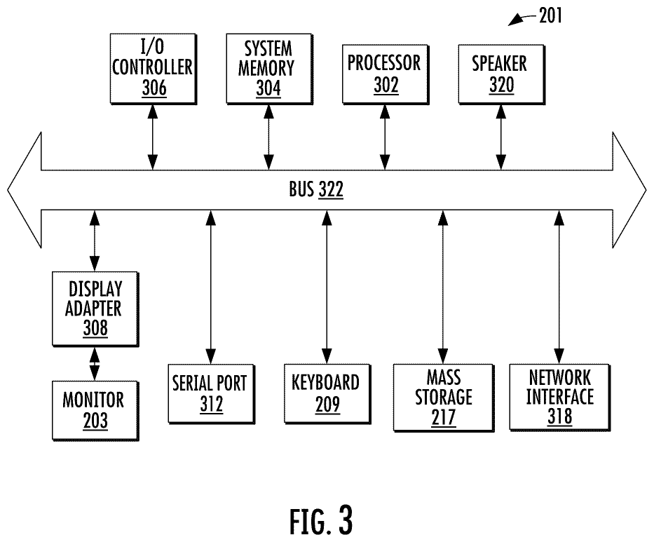

FIG. 3 shows a system block diagram of computer system 201 used to execute the software of the present invention. As in FIG. 2, computer system 201 includes monitor 203, keyboard 209, and mass storage devices 217. Computer system 501 further includes subsystems such as central processor 302, system memory 304, input/output (I/O) controller 306, display adapter 308, serial or universal serial bus (USB) port 312, network interface 318, and speaker 320. The invention may also be used with computer systems with additional or fewer subsystems. For example, a computer system could include more than one processor 302 (i.e., a multiprocessor system) or a system may include a cache memory.

Arrows such as 322 represent the system bus architecture of computer system 201. However, these arrows are illustrative of any interconnection scheme serving to link the subsystems. For example, speaker 320 could be connected to the other subsystems through a port or have an internal direct connection to central processor 302. The processor may include multiple processors or a multicore processor, which may permit parallel processing of information. Computer system 201 shown in FIG. 2 is but an example of a computer system suitable for use with the present invention. Other configurations of subsystems suitable for use with the present invention will be readily apparent to one of ordinary skill in the art.

Computer software products may be written in any of various suitable programming languages, such as C, C++, C#, Pascal, Fortran, Perl, Matlab (from MathWorks, www.mathworks.com), SAS, SPSS, JavaScript, AJAX, Java, Python, Erlang, and Ruby on Rails. The computer software product may be an independent application with data input and data display modules. Alternatively, the computer software products may be classes that may be instantiated as distributed objects. The computer software products may also be component software such as Java Beans (from Oracle Corporation) or Enterprise Java Beans (EJB from Oracle Corporation).

An operating system for the system may be one of the Microsoft Windows® family of systems (e.g., Windows 95, 98, Me, Windows NT, Windows 2000, Windows XP, Windows XP x64 Edition, Windows Vista, Windows 7, Windows 8, Windows 10, Windows 11, Windows CE, Windows Mobile, Windows RT), Symbian OS, Tizen, Linux, HP-UX, UNIX, Sun OS, Solaris, Mac OS X, Apple iOS, Android, Alpha OS, AIX, IRIX32, or IRIX64. Other operating systems may be used. Microsoft Windows is a trademark of Microsoft Corporation.

Furthermore, the computer may be connected to a network and may interface to other computers using this network. The network may be an intranet, internet, or the Internet, among others. The network may be a wired network (e.g., using copper), telephone network, packet network, an optical network (e.g., using optical fiber), or a wireless network, or any combination of these. For example, data and other information may be passed between the computer and components (or steps) of a system of the invention using a wireless network using a protocol such as Wi-Fi (IEEE standards 802.11, 802.11a, 802.11b, 802.11e, 802.11g, 802.11i, 802.11n, 802.11ac (e.g., Wi-Fi 5), 802.11ad, 802.11ax (e.g., Wi-Fi 6), and 802.11af, just to name a few examples), near field communication (NFC), radio-frequency identification (RFID), mobile or cellular wireless (e.g., 2G, 3G, 4G, 5G, 3GPP LTE, WiMAX, LTE, LTE Advanced, Flash-OFDM, HIPERMAN, iBurst, EDGE Evolution, UMTS, UMTS-TDD, 1xRDD, and EV-DO). For example, signals from a computer may be transferred, at least in part, wirelessly to components or other computers.

In an embodiment, with a web browser executing on a computer workstation system, a user accesses a system on the World Wide Web (WWW) through a network such as the Internet. The web browser is used to download web pages or other content in various formats including HTML, XML, text, PDF, and postscript, and may be used to upload information to other parts of the system. The web browser may use uniform resource identifiers (URLs) to identify resources on the web and hypertext transfer protocol (HTTP) in transferring files on the web.

In other implementations, the user accesses the system through either or both of native and nonnative applications. Native applications are locally installed on the particular computing system and are specific to the operating system or one or more hardware devices of that computing system, or a combination of these. These applications (which are sometimes also referred to as “apps”) can be updated (e.g., periodically) via a direct internet upgrade patching mechanism or through an applications store (e.g., Apple iTunes and App store, Google Play store, Windows Phone store, and Blackberry App World store).

The system can run in platform-independent, nonnative applications. For example, client can access the system through a web application from one or more servers using a network connection with the server or servers and load the web application in a web browser. For example, a web application can be downloaded from an application server over the Internet by a web browser. Nonnative applications can also be obtained from other sources, such as a disk.

Medical imaging currently renders patient x-rays, MRIs, ultrasound, and mammogram images via hardware, and very little software is used to matriculate the capture signal into a clear, readable image for a radiologist. Specific physical electronic components are selected to attempt to provide an optimal image output. If any component is changed or upgraded, the entire system requires manual recalibration or system optimization. Furthermore, once an image is available for the radiologist from a given device, all digital analytics, deep-learning analysis is executed outside the medical device such as at a reading workstation, an on-premises computation system, or the IT network or cloud. This post-capture analysis can cause latency and delay diagnosis for the patient.

The following are incorporated by reference: U.S. patent application Ser. No. 18/625,028, filed Apr. 2, 2024, which is a continuation of U.S. patent application Ser. No. 17/305,864, filed Jul. 15, 2021, issued as U.S. Pat. No. 11,948,297 on Apr. 2, 2024, which claims the benefit of U.S. patent application 63/052,411, filed Jul. 15, 2020.

### Image Enhancement and Embedding Deep Learning-Based Algorithms in Medical Imaging Equipment

An example where deep learning technique is used in the medical field is mammography. Mammography is used to screen for breast cancer and other breast abnormalities. Traditionally, mammograms were done using x-ray films, however recently digital x-ray imaging is used to capture the breast images. The digital images facilitate easy storage and analysis. Digital images make it easier for applying Deep Learning models for assessment and prediction of cancer. Three-dimensional (3D) mammogram is the latest development in mammography, also called digital breast tomosynthesis (DBT). Two-dimensional mammography is a full-field digital mammography (FFDM), and synthetic 2D mammography produces 2D pictures derived from 3D data produced by DBT by combining various enhanced slices of DBT. Breast tomosynthesis reconstructs a 3D image out of various 2D images obtained as a series of projected x-ray images obtained by angular displacement of an x-ray source.

Another modality for breast imaging is ultrasound. Ultrasound is a high frequency soundwave; the reflected soundwaves containing information on the depth, kind of tissue or fluid are collected, and an image is reconstructed. Ultrasound imaging of the breast is used when the breast density is high and filled with glandular and connective tissue and less fatty muscles, making it difficult to see the tumor in an x-ray or an MRI image. These ultrasound images can also be used with Deep Learning models to predict breast cancer.

Yet another modality for breast imaging is Magnetic Resonance Imaging (MRI). MRI uses magnetic waves to produce images of the internal organs. Breast MRI produces images of breast tissue by detecting movements of atoms within the body which can reveal information about the abnormalities of the breast. Deep Learning models can also be used on MRI images to classify the images as cancer or benign.

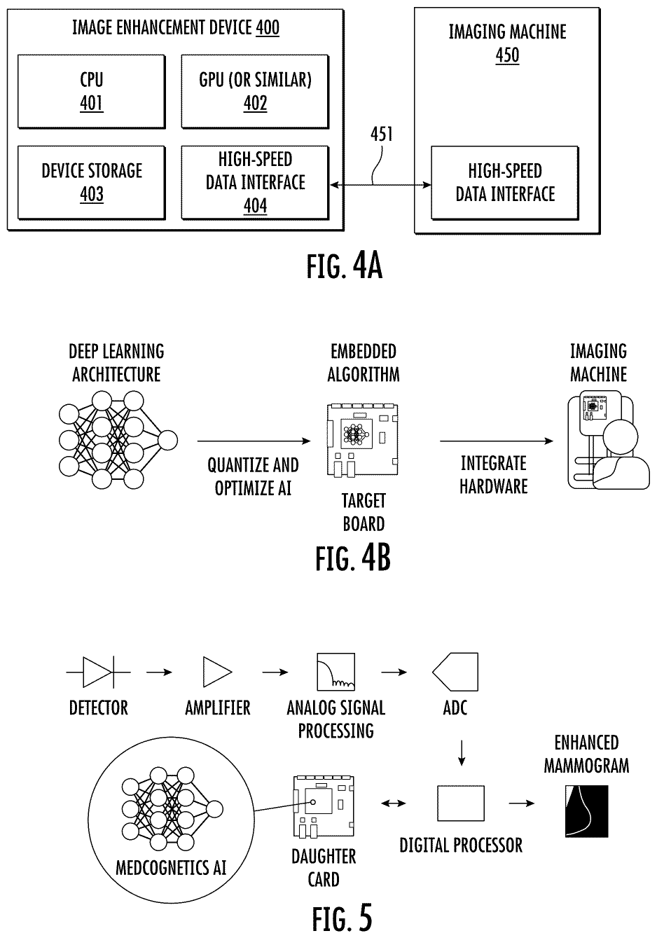

FIG. 4a describes the embedded image processing system inside an imaging machine incorporating an embodiment of the present invention. The embodiment consists of Image enhancement device (400) connected to an imaging machine (450). The image enhancement device (400) consists of a central processing unit (401), a processing unit for accelerated inference of artificial intelligence (AI) algorithms and associated mathematics (402), an electronic memory for one or more artificial intelligence algorithms storage (403) and a high-speed data transfer interface (404). Data for processing is transferred from an imaging machine (450) to the image enhancement device over a high-speed interface (404). The image enhancement device (400) loads algorithms from the device storage (403) and processes patient data with the CPU (401) and mathematics accelerator (402). Once the raw data has been enhanced, it is returned to the imaging machine (450) through the high-speed interface (451) or directly to a machine workstation computer.

FIG. 4b illustrates Artificial Intelligence (AI) software being embedded into a semiconductor chip, the semiconductor chip is placed on an electronic circuit board and said circuit board is installed physically inside the medical imaging machine described by FIG. 4a.

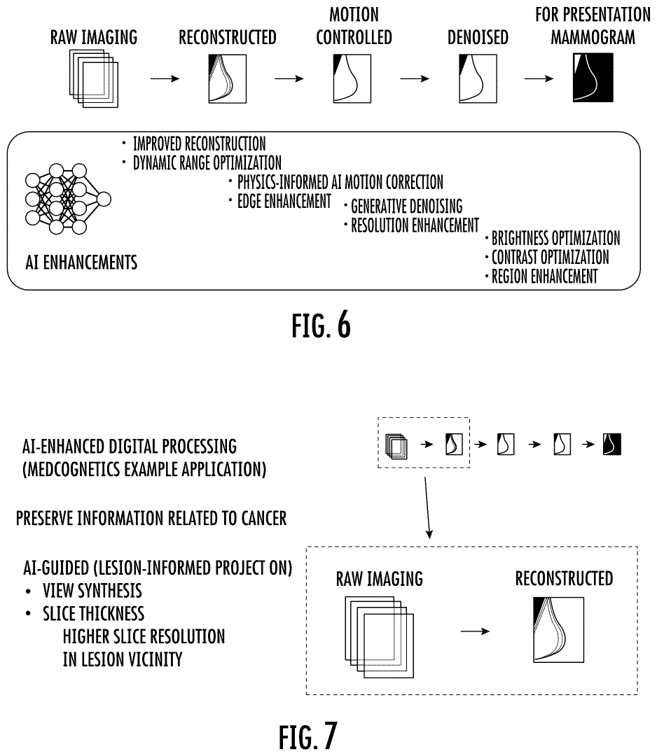

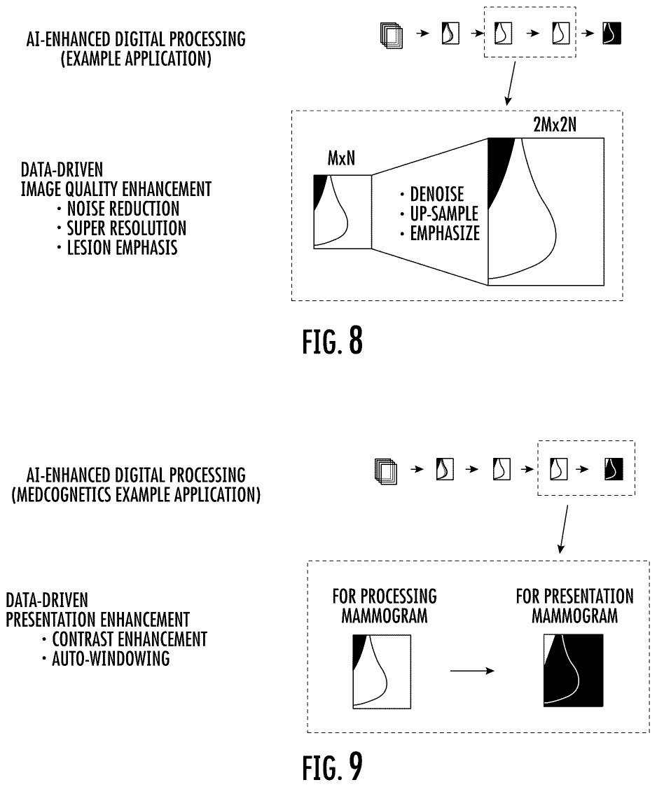

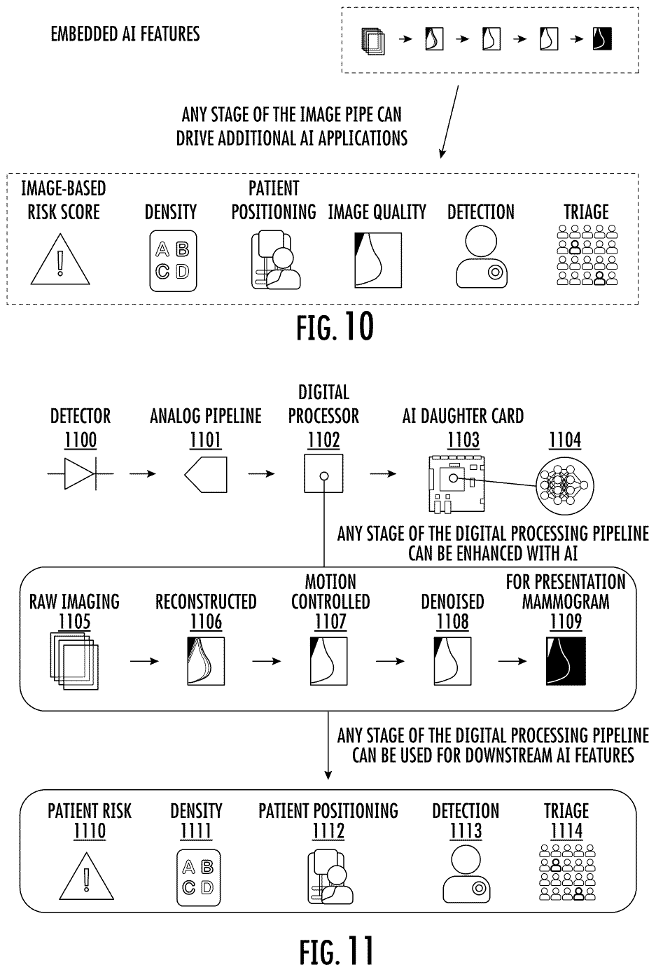

FIG. 5 provides context to where within the analog and digital hardware signal chain for the image processing path, or “pipe” the artificial intelligence software (digital processor) is located. Such software can have features and functions as depicted in FIG. 6. Some software defined imaging enhancement features and functions are depicted in FIGS. 7, 8, and 9. Any of these embedded imaging features in the image pipe can have artificial intelligence analysis software implemented in these functional blocks of image processing. FIG. 10 depicts additional artificial intelligence applications that can the served from the embedded image processing system.

FIG. 11 represents the full embodiment of our system, 1100 is the analog wave signal detector on a medical device for either x-ray, MRI, or ultrasound. This analog receive signal has passed through a portion of a patient's anatomy. This analog signal is converted to a digital signal in 1101, which then can be put into a digital signal processor, 1102 which physically resides on an electronic circuit board, 1103. It should be noted that 1100, 1101, 1102, and 1103 are hardware components of this system, the medical imaging device, specifically in this example we are depicting mammogram gantry hardware. The embedded software for image processing and artificial intelligence analysis for cancer related factors is 1104. The raw image data that comes from the analog pipeline, 1101, is represented in FIG. 11 as 1105. The raw image, 1105, is passed through the embedded software 1104, which has two functional areas, artificial intelligence image processing/enhancement (1106, 1107, 1108, and 1109) and artificial intelligence image analysis (1110, 1111, 1112, 1113, and 1114) that have individual sub-functions as listed in the parenthesis. The artificial intelligence image analysis features (1110, 1111, 1112, 1113, and 1114) can be implemented anywhere along the digital processing pipeline as depicted by 1105, 1106, 1107, 1108, and 1109.

The enhanced reconstruction of the image is done in 1106 and the algorithm used for the enhanced reconstruction is given below:

### Algorithm I. Projection Synthesis

In DBT, N number of x-ray projections are captured per breast. A higher number of projections, than N, can improve final volume quality but at the cost of time and higher radiation exposure which may be detrimental to the patient. However, by using artificial intelligence, which has been trained across many patients, an additional M number of projections can be synthesized using generative artificial intelligence without altering standard patient radiation exposure. The N+M projections derived from sensor data and artificial intelligence algorithm will produce a better-quality image volume than N projections alone. An example training strategy for the artificial intelligence model that can provide the projections, using either real or simulated patient data, is presented below. This is one of many similar strategies which can be used to train our algorithm.
1. Randomly remove 1 projection from N captured projections
2. Provide N−1 remaining projections to the artificial intelligence architecture
3. Provide the positions of the N−1 remaining projections to the artificial intelligence architecture
4. Provide the position of the removed projection to the artificial intelligence architecture
5. Compare the output of the artificial intelligence algorithm for the removed projection and calculate a loss value
6. Train the artificial intelligence architecture based on the loss calculated in the previous step
7. Repeat steps 1 through 6 until the artificial intelligence architecture is fully trained

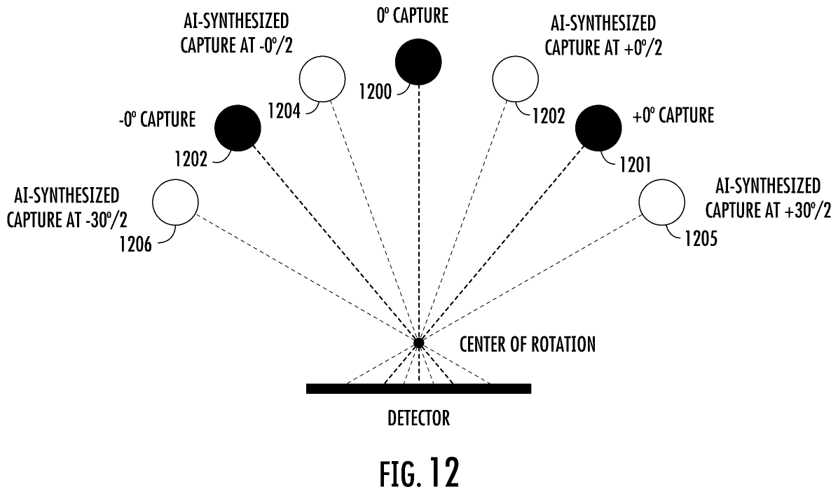

During deployment, this deep neural network architecture can predict several M additional projections as shown in FIG. 12. The image projection at 0 degree is shown as (1200), the image projection +θ degree is denoted as 1201 and now the artificial intelligence synthesized projection of the image at +θ/2 degree is shown as (1203). Similarly, the artificial intelligence synthesized projection of the image at −θ/2 (1205) degree is computed using the artificial intelligence algorithm and the projections at 0 degree (1200) and −θ degree (1204). Angles such as +3θ/2, beyond the capture angles, may be synthesized by artificial intelligence as well. These additional M projections can create a higher-quality DBT volume when combined with the N projections captured via sensor data. The N+M projections are created for the radiation exposure cost of N projections.

The deep learning architecture used for this algorithm is a vision transformer. Vision transformer provides attention, a mathematical mechanism by which information from multiple projections may be directly leveraged in determining a new projection. A vision transformer also includes convolutional neural network layers for translating raw pixel values into feature-based representations. Other than vision transformer, convolutional neural networks architectures can also be used. For example, Convnext is one of the convolutional neural network architectures that can be used.

### Algorithm II. Voxel Predictions

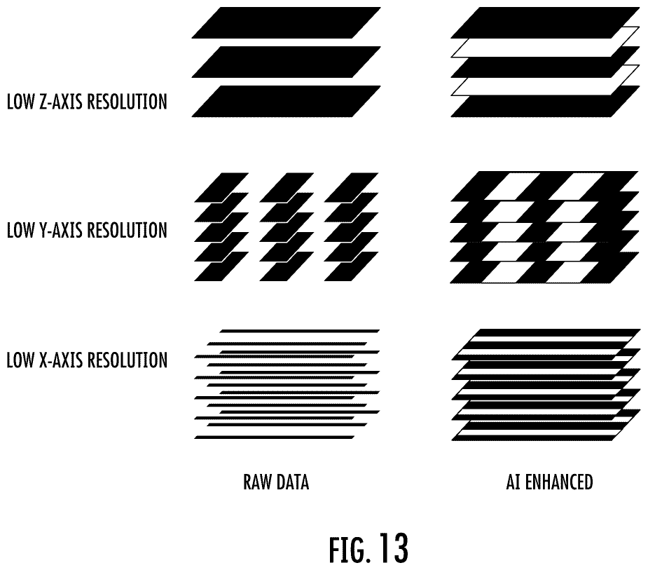

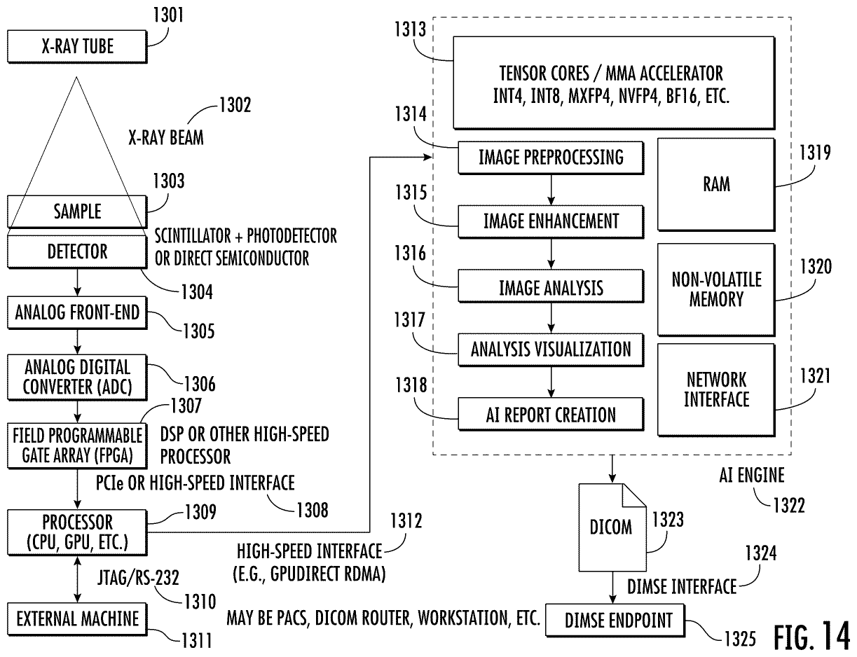

In DBT, N number of voxels (3D-pixels) are captured per breast. A higher number of voxels, N, can improve final volume quality by increasing volume resolution. By using artificial intelligence trained across many patients, an additional M number of voxels can be synthesized without altering standard patient radiation exposure. The N+M voxels derived from sensor data plus artificial intelligence will produce a better-quality image volume than N voxels alone. An example training strategy, using either real or simulated patient data, is presented below. This is one of many similar strategies which can be used to train our system.
8. Randomly remove X voxels from N captured voxels
9. Provide N−X remaining voxels to the artificial intelligence architecture
10. Provide the positions of the N−X remaining voxels to the artificial intelligence architecture
11. Provide the positions of the removed voxels to the artificial intelligence architecture
12. Compare the output of the AI architecture to the removed voxels and calculate a loss value
13. Train the artificial intelligence architecture based on the loss calculated in the previous step
14. Repeat steps 1 through 6 until the artificial intelligence architecture is fully trained During deployment, this architecture can be used to predict several M additional voxels at number M different voxel positions. These additional M voxels can create a higher-resolution DBT volume as shown in FIG. 13 when combined with the N voxels computed from sensor data. Like algorithm I, projection synthesis, the deep learning architecture for this algorithm may be like the architecture of a vision transformer. Vision transformer provides attention, a mathematical mechanism by which information from multiple projections may be directly leveraged in determining a new projection. A vision transformer may also include convolutional neural network layers for translating raw pixel values into feature-based representations. Other than vision transformer, convolutional neural networks architectures can also be used. For example, Convnext is one of the convolutional neural network architectures that can be used.

The motion noise is controlled in module 1107 and all other noises including scattering noise and sensor noise are removed in module 1108. A deep learning described below as Algorithm III is used in modules 1107 and 1108 for noise removal.

### Algorithm III. Noise (Motion, Scattering, Sensor) Removal

The third algorithm used by this system removes sources of noise including sensor, scatter, and motion noise. This algorithm may be applied on raw projections or reconstructed slices. Training data for this deep learning algorithm is sourced through the following approach:
15. Producing data samples with/without noise via simulation
15a. Simulation works with any noise source that can be physically modeled
15b. Simulation creates ideal reference studies with noise completely absent

The other techniques used for generating training data are:
16. Capturing real data with/without noise by imaging inorganic phantom objects.
16a. Phantoms eliminate patient risk from extended or repeated imaging in a brief time.
16b. Phantoms may be created through injection molding, 3D printing, and other common manufacturing processes
16c. Phantom designs may be based on simulated models or data captured from real patients
16d. For certain noise such as motion noise, additional mechanical control and/or noise generating mechanisms may be necessary
17. Using real data captured accidentally with excessive noise during imaging.
17a. Some patients undergo additional imaging because the original images contained excessive amounts of noise (e.g., too much patient motion)
17b. Leveraging this data does not provide any additional unnecessary risk to patients

Like algorithms I and II, the deep learning architecture for this algorithm is the architecture of a vision transformer. Additionally, a diffusion model and U-Net like architecture are also applicable. A vision transformer will be able to apply attention across multiple projections, frames, and/or slices while denoising, whereas diffusion and U-Net-like models will be more effective at image-by-image processing. In the place of or in conjunction with vision transformers, diffusion model and U-net like architectures, convolutional neural networks architectures can also be used. For example, Convnext is one of the convolutional neural network architectures that can be used.

FIG. 14 shows a system diagram of an imaging machine with embedded image processing system. A medical image is created via x-ray (or MRI, CT Scan, PET Scan, PET-CT Scan, Ultrasound, and other medical imaging devices) using a hardware configuration as shown by several hardware modules 1301-1309. This medical image (or images or 3D scan) is sent to an AI engine 1322 using a high-speed interface 1312 such as GPUDirect RDMA. The AI engine 1322 uses tensors cores or matrix multiply accumulate (MMA) accelerator 1313 as provided by an application specific integrated circuit (ASIC), field programmable gate array (FPGA), central processing unit (CPU), graphics processing unit (GPU), controller, state machine, or others to perform various algorithmic stages: image preprocessing, image enhancement, image analysis, analysis visualization, and AI report creation, such as described above. A final output of the AI engine 1322 is provided as a Digital Imaging and Communications in Medicine (DICOM) (or other format) to downstream workflows.

Specifically, hardware and related components include an x-ray tube 1301, x-ray beam 1302, sample for examination 1303, detector 1304 (which can be a scintillator and photodetector or direct semiconductor), analog front end or AFE 1305, analog-to-digital converter (ADC) 1306, field programmable gate array (FPGA) or DSP or other high-speed processor 1307, PCI Express (PCIe) or other high-speed interface or bus 1308, and processor (e.g., CPU, GPU, and others) 1309.

PCI Express (Peripheral Component Interconnect Express), officially abbreviated as PCIe, is a high-speed standard used to connect hardware components inside computers or computing systems. It is designed to replace older expansion bus standards such as PCI, PCI-X and AGP. Developed and maintained by the PCI Special Interest Group or PCI-SIG, PCIe is commonly used to connect graphics cards, sound cards, Wi-Fi and Ethernet adapters, and storage devices such as solid-state drives and hard disk drives. PCI Express cards can be peripheral cards or daughter cards that can expand the functionality of an existing computer or system to have additional features not available through only the motherboard on which the PCIe connectors are situated. For example, a GPU card can be added to system via a PCIe or other connector to accelerate graphics of the system. A medical image processor 1309 can be added to system via a PCIe or other connector or port (e.g., USB or Thunderbolt port) of an existing medical imaging system to expand its capabilities. These capabilities can include accelerated or improved medical image processing or diagnosis, or a combination. Further, these capabilities can include diagnosis using an artificial intelligence engine using techniques as described in this patent.

Further the processor is coupled via a JTAG or RD-232 or other interface or bus 1310 to an external machine 1311. The processor can include or be connected to an artificial intelligence (AI) engine, which may be connected to a medical imaging system via PCIe or other connector or port (e.g., USB or Thunderbolt port). The AI engine can include tensor cores or MMA accelerator (e.g., INT4, INT8, MXFP4, NVFP4, BF16, and others) 1334, image preprocessing 1314, image enhancement 1315, image analysis 1316, analysis visualization 1317, artificial intelligence (AI) report creation 1318, volatile memory (e.g., RAM) 1319, Nonvolatile memory 1320 (e.g., Flash or electrically erasable memory), and network interface 1321. An output of the processor is a DICOM 1323 file. The DICOM file can be transferred via a DIMSE interface 1324 to a DIMSE endpoint 1325, such as a PACS, DICOM router, workstation, and others.

DICOM is a technical standard for the digital storage and transmission of medical images and related information. It includes a file format definition, which specifies the structure of a DICOM file, as well as a network communication protocol that uses TCP/IP to communicate between systems. The primary purpose of the standard is to facilitate communication between the software and hardware entities involved in medical imaging, especially those that are created by different manufacturers. Entities that utilize DICOM files include components of picture archiving and communication systems (PACS), such as imaging machines (modalities), radiological information systems (RIS), scanners, printers, computing servers, and networking hardware.

Medical imaging is the technique and process of imaging the interior of a body for clinical analysis and medical intervention, as well as visual representation of the function of some organs or tissues (physiology). Medical imaging seeks to reveal internal structures hidden by the skin and bones, as well as to diagnose and treat disease. Medical imaging also establishes a database of normal anatomy and physiology to make it possible to identify abnormalities. Although imaging of removed organs and tissues can be performed for medical reasons, such procedures are usually considered part of pathology instead of medical imaging.

An x-ray machine is a device that uses x-rays for a variety of applications including medicine, x-ray fluorescence, electronic assembly inspection, and measurement of material thickness in manufacturing operations. In medical applications, x-ray machines are used by radiographers to acquire x-ray images of the internal structures (e.g., bones) of living organisms, and also in sterilization.

An x-ray generator generally contains an x-ray tube to produce the x-rays. Possibly, radioisotopes can also be used to generate x-rays. An x-ray tube is a simple vacuum tube that contains a cathode, which directs a stream of electrons into a vacuum, and an anode, which collects the electrons and is made of tungsten to evacuate the heat generated by the collision. When the electrons collide with the target, about 1 percent of the resulting energy is emitted as x-rays, with the remaining 99 percent released as heat. Due to the high energy of the electrons that reach relativistic speeds, the target is usually made of tungsten even if other material can be used particularly in XRF applications. An x-ray generator can also to contain a cooling system to cool the anode. For example, many x-ray generators use water or oil recirculating systems.

In medical imaging applications, an x-ray machine has a control console that is used by a radiologic technologist to select x-ray techniques suitable for the specific exam, a power supply that creates and produces the desired kVp (peak kilovoltage), mA (milliamperes, sometimes referred to as mAs which is actually mA multiplied by the desired exposure length) for the X-ray tube, and the x-ray tube itself.

A computed tomography scan (CT scan), formerly called computed axial tomography scan (CAT scan), is a medical imaging technique used to obtain detailed internal images of the body. CT scanners use a rotating x-ray tube and a row of detectors placed in a gantry to measure x-ray attenuations by different tissues inside the body. The multiple x-ray measurements taken from different angles are then processed on a computer using tomographic reconstruction algorithms to produce tomographic (cross-sectional) images (virtual “slices”) of a body. CT scans can be used in patients with metallic implants or pacemakers, for whom magnetic resonance imaging (MRI) is contraindicated.

Positron emission tomography-computed tomography (better known as PET-CT or PET/CT) is a nuclear medicine technique which combines, in a single gantry, a positron emission tomography (PET) scanner and an x-ray computed tomography (CT) scanner, to acquire sequential images from both devices in the same session, which are combined into a single superposed (co-registered) image. Thus, functional imaging obtained by PET, which depicts the spatial distribution of metabolic or biochemical activity in the body can be more precisely aligned or correlated with anatomic imaging obtained by CT scanning. Two- and three-dimensional image reconstruction may be rendered as a function of a software and control system.

Magnetic resonance imaging (MRI) is a medical imaging technique used in radiology to generate pictures of the anatomy and the physiological processes inside the body. MRI scanners use strong magnetic fields, magnetic field gradients, and radio waves to form images of the organs in the body. MRI does not involve X-rays or the use of ionizing radiation, which distinguishes it from computed tomography (CT) and positron emission tomography (PET) scans. MRI is a medical application of nuclear magnetic resonance (NMR) which can also be used for imaging in other NMR applications, such as NMR spectroscopy.

Medical ultrasound includes diagnostic techniques (e.g., imaging) using ultrasound. In diagnosis, it is used to create an image of internal body structures such as tendons, muscles, joints, blood vessels, and internal organs, to measure some characteristics (e.g., distances and velocities) or to generate an informative audible sound. Ultrasound is composed of sound waves with frequencies greater than 20,000 Hz, which is the approximate upper threshold of human hearing. Ultrasonic images are created by sending pulses of ultrasound into tissue using a probe. The ultrasound pulses echo off tissues with different reflection properties and are returned to the probe which records and displays them as an image.

The usage of ultrasound to produce visual images for medicine is called medical ultrasonography or simply sonography. Sonography using ultrasound reflection is called echography. There are also transmission methods, such as ultrasound transmission tomography. The practice of examining pregnant women using ultrasound is called obstetric ultrasonography, and was an early development of clinical ultrasonography. The machine used is called an ultrasound machine, a sonograph or an echograph. The visual image formed using this technique is called an ultrasonogram, a sonogram or an echogram.

An ultrasound imaging mode refers to probe and machine settings that result in specific dimensions of the ultrasound image. Several modes of ultrasound are used in medical imaging including:
A-mode: Amplitude mode refers to the mode in which the amplitude of the transducer voltage is recorded as a function of two-way travel time of an ultrasound pulse. A single pulse is transmitted through the body and scatters back to the same transducer element. The voltage amplitudes recorded correlate linearly to acoustic pressure amplitudes. A-mode is one-dimensional.
B-mode: In brightness mode, an array of transducer elements scans a plane through the body resulting in a two-dimensional image. Each pixel value of the image correlates to voltage amplitude registered from the backscattered signal. The dimensions of B-mode images are voltage as a function of angle and two-way time.
M-mode: In motion mode, A-mode pulses are emitted in succession. The backscattered signal is converted to lines of bright pixels, whose brightness linearly correlates to backscattered voltage amplitudes. Each next line is plotted adjacent to the previous, resulting in an image that looks like a B-mode image. The M-mode image dimensions are however voltage as a function of two-way time and recording time. This mode is an ultrasound analogy to streak video recording in high-speed photography. As moving tissue transitions produce backscattering, this can be used to determine the displacement of specific organ structures, most commonly the heart.

In an implementation, an embedded processor containing deep learning algorithms capable of delivering one or more image features by leveraging intermediate representations at any stage of the digital processing pipeline inside the imaging machine. The deep learning algorithm is composed of fully connected, attention, convolutional, or any layers performing linear and non-linear operations for feature extraction and signal processing, or any combination. The deep learning algorithm can denoise the image by removing optical artifacts, scatter noise, and any other unwanted signal. The deep learning algorithm can filter motion control to remove image blurring due to patient or body movement at the instance of image capture. The algorithm for analyzing the mammogram, where layers of a deep learning network extract features for the context of an optimizing or correcting the position of the patient who is being imaged, or a combination. The algorithm can predict the breast density from the image. The algorithm for analyzing mammogram, where layers of a deep learning network extract image features for the context of a specific cancer or benign outcome for a triage case list for presentation to the radiologist. The algorithm can denote the region of interest (RoI) of cancer in the mammogram image. The algorithm can assess the risk of the patient getting breast cancer from the breast image and provide a risk score. The algorithm is capable of increasing the image clarity and resolution without the physical increase of the projections or voxels in image capture. The algorithm can be based on projection synthesis using generative AI. The algorithm can be based on voxel synthesis using generative AI.

In an implementation, a system for generating an artificial-intelligence-enhanced medical image output, including: an imaging device configured to acquire a medical image of a subject using at least one imaging modality selected from the group consisting of X-ray, magnetic resonance imaging (MRI), and ultrasound; a hardware interface configured to transmit the medical image from the imaging device over a high-speed data pathway including a remote direct memory access (RDMA) protocol; an AI engine in communication with the hardware interface, the AI engine including one or more tensor-processing cores or matrix-multiply-accumulate (MMA) accelerators implemented on at least one of an application-specific integrated circuit (ASIC), a field-programmable gate array (FPGA), or a graphics processing unit (GPU); and where the AI engine is configured to execute a sequence of algorithmic stages including: (i) image preprocessing; (ii) image enhancement; (iii) image analysis; (iv) analysis visualization; and (v) automated AI report generation; and where the AI engine is further configured to generate a final output in a DICOM-compliant format or another medical-image format for delivery to a downstream clinical workflow.

In an implementation, a method for generating an AI-derived medical imaging output, the method including: acquiring, with an imaging device, at least one medical image of a subject using X-ray, MRI, ultrasound, or another medical imaging modality; transmitting the medical image to an artificial-intelligence engine over a high-speed interface implementing a remote direct memory access (RDMA) transfer protocol; processing the medical image within the artificial-intelligence engine using one or more tensor cores or matrix-multiply-accumulate (MMA) accelerators implemented on an ASIC, FPGA, or GPU; executing, within the artificial-intelligence engine, a plurality of algorithmic stages including image preprocessing, image enhancement, image analysis, analysis visualization, and automated report creation; and outputting a final processed medical image or analysis in a DICOM format or another medical imaging format for use in a downstream workflow.

In an implementation, a nontransitory computer-readable medium storing instructions which, when executed by one or more processors including tensor-processing cores or matrix-multiply-accumulate (MMA) accelerators implemented on an ASIC, FPGA, or GPU, cause the processors to: receive a medical image acquired by an imaging device using X-ray, MRI, ultrasound, or another medical imaging modality via a high-speed hardware interface utilizing a remote direct memory access (RDMA) transfer mechanism; perform image preprocessing, image enhancement, image analysis, analysis visualization, and automated AI report generation on the medical image; and produce a final output in a DICOM-compliant format or another medical-image format for transfer to downstream medical workflows.

In an implementation, an artificial-intelligence apparatus for processing medical images, including: one or more accelerators including tensor cores or matrix-multiply-accumulate (MMA) units implemented on an ASIC, FPGA, or GPU; a high-speed memory interface configured to receive medical imaging data via RDMA; and control logic configured to execute a multi-stage processing pipeline including image preprocessing, image enhancement, image analysis, visualization of analysis results, and automated generation of a medical report; where the apparatus outputs the processed image or report in a DICOM-compliant or other medical-image format.

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

What is claimed is:

1. A system comprising:
a medical imaging emitter device;
a medical imaging detector device, wherein a tissue to be examined will be placed in between a signal path from the emitter device to the detector device;
an analog front end circuit, coupled to the detector circuit;
an analog-to-digital converter (ADC) circuit, coupled to the detector circuit;
a digital signal processing (DSP) circuit, coupled to the analog-to-digital converter circuit;
a system bus, coupled to digital signal processing circuit;
a processor, coupled to system bus; and
an image processing circuit, coupled to the system bus, wherein the image processing circuit comprises:
a plurality of tensor cores or matrix-multiply-accumulate units;
a random access memory, coupled to the tensor cores or matrix-multiply-accumulate units;
a nonvolatile memory, coupled to the tensor cores or matrix-multiply-accumulate units; and
a network interface; coupled to the tensor cores or matrix-multiply-accumulate units.

2. The system of claim 1 wherein the digital signal processing (DSP) circuit comprises a field programmable gate array.

3. The system of claim 1 wherein an image processing circuit is configured to execute (i) image preprocessing, (ii) image enhancement, (iii) image analysis, (iv) analysis visualization, and (v) automated report generation.

4. The system of claim 1 wherein the image processing circuit is configured to execute a fully connected, attention, convolutional, or any layers performing linear and non-linear operations for feature extraction and signal processing, or any combination.

5. The system of claim 1 wherein the medical imaging emitter device comprises an x-ray emitter.

6. The system of claim 1 wherein the medical imaging emitter device comprises a magnetic field emitter.

7. The system of claim 1 wherein the medical imaging emitter device comprises a positron emitter.

8. The system of claim 1 wherein an image processing circuit is configured to execute one or more deep learning algorithms capable of delivering one or more image features by leveraging intermediate representations at any stage of the digital processing pipeline inside the imaging machine.

9. The system of claim 8 comprising of fully connected, attention, convolutional, or any layers performing linear and non-linear operations for feature extraction and signal processing, or any combination.

10. The system of claim 8 comprising denoising the image by removing optical artifacts, scatter noise, and any other unwanted signal.

11. The system of claim 8 comprising filtering motion control to remove image blurring due to patient or body movement at the instance of image capture.

12. The system of claim 8, wherein the image of the tissue is a mammogram, and layers of a deep learning network extract features for the context of an optimizing or correcting the position of the patient who is being imaged, or a combination.

13. The system of claim 8 comprising predicting a breast density from the image.

14. The system of claim 8, wherein the image of the tissue is a mammogram, and layers of a deep learning network extract image features for the context of a specific cancer or benign outcome for a triage case list for presentation to the radiologist.

15. The system of claim 8 comprising denoting a region of interest (RoI) of cancer in a mammogram image.

16. The system of claim 8 comprising assessing the risk of the patient getting breast cancer from the breast image and provide a risk score.

17. The system of claim 8 comprising increasing an image clarity and resolution without the physical increase of the projections or voxels in image capture.

18. The system of claim 8 comprising increasing an image clarity and resolution based on projection synthesis using a generative intelligence technique, without the physical increase of the projections or voxels in image capture.

19. The system of claim 8 comprising increasing an image clarity and resolution based on voxel synthesis using a generative intelligence technique.
